(19) United States
(12) Patent Application Publication
(10) Pub. No.: US 2011/0207155 A1
(43) Pub. Date: Aug. 25, 2011

(54) Method for the preparation of immunoconjugates and use thereof

(75) Inventors: Paolo Pengo, Venezia Marghera (IT); Luca Beneduce, Venezia Marghera (IT); Giorgio Fassina, Venezia Marghera (IT)

(73) Assignee: XEPTAGEN SPA, Venezia Marghera (IT)

(21) Appl. No.: 13/121,407

(22) PCT Filed: Oct. 13, 2008
(86) PCT No.: PCT/IB2008/054203

§ 371 Date: May 6, 2011

(51) Int. Cl. G01N 33/53; C07K 1/00; C12N 9/96

(52) U.S. Cl. 435/7.92; 530/391.1; 530/409; 435/188; 436/501

(57) ABSTRACT

The present invention concerns a method for the preparation of molecular conjugates comprising immunoglobulins and biomarkers occurring in neoplastic diseases characterized by high levels of reproducibility.

The present patent describes a method for the synthesis of molecular species and namely a method for the synthesis of conjugates and a new method to optimize the reactivity of said conjugates and the use thereof in procedures pertinent to oncology diagnostics. Tumors represent nowadays the second leading cause of death after cardiovascular diseases and their incidence rate is increasing worldwide. Although the distribution and the incidence of each specific type of cancer are very different when different world regions are concerned, said incidence rates show very similar trends in industrialized countries. In these countries, the neoplastic diseases of the lungs and prostate gland represent a large part of the reported cancer cases within the male population. Within the female population a large part of the reported cancer cases are breast cancer, while the incidence of lung cancer is increasing. Besides these gender-specific tumors, the incidence rates of intestinal cancer and pancreatic carcinoma are continuously increasing, in facts, they doubled in the last 50 years. From these considerations clearly stems the importance of the quest for specific markers able to signal the neoplastic transformation with high organ-specificity. The presence of tumors often produces a specific molecular signature as the result of the altered metabolism in those cells that underwent neoplastic transformation. The presence of these substances, known as tumor biomarkers or tumor associated antigens, may be detected exploiting histological methods or, more conveniently from the clinical point of view, by means of analyses carried out on biological fluids such as serum or plasma. However, in some cases, the modification of the normal biomarker profile may not be an useful index of the presence of neoplastic diseases. This occurs because these modifications may be the result of lifestyle, non neoplastic diseases or to be iatrogenic (Gion, M. in : “Guida all'utilizzo dei biomarcatori in oncologia” Biomedia Source Books edition, Milan 2002, ITALY). From the diagnostic point of view, this is a source of false-positive results within a cohort of healthy individuals.

The same applicant owns a patent application concerning an highly specific method for the diagnosis of cancer, where the identification of the biomarker does not give indication of a pathologic condition in healthy subjects. In closer details: the applicant devised tests capable to attain a better correlation between the presence of a biomarker and the pathologic condition of the subjects. These tests have been developed from the experimental evidence that in sera of patients affected by neoplastic diseases, besides the presence of free biomarkers, i.e. biomarkers not conjugated to other co-factors, other biomarker containing species may be detected. The latter species are complexes of said biomarkers with immunoglobulins, namely immunoglobulins of the M class or IgM. Hence, a method based on the identification of the circulating immunocomplexes formed by the association of tumor marker and autoantibody specific thereto, hereinafter referred to as specific CIC, has been conceived. This method is extremely useful in cancer diagnosis, since it allows the discrimination of healthy subjects from a cohort of patients affected by cancer with higher selectivity with respect to tests based on the detection of the free tumor antigen. Autoantibodies are antibodies naturally produced by immune system and directed against an antigen recognized as non-self, even though it is one of the molecules normally expressed by the organism. The common explanation for the immunogenicity of these self species is related to their anomalously high expression (hyperexpression) in tissues that underwent neoplastic transformation when compared to normal tissues (Sahin U. et al. Proc. Natl. Acad. Sci. USA, 1995, 92, 11810-13). For the reasons outlined above, the analysis of the free tumor antigens may have poor diagnostic value, especially when high specificities are concerned. Instead, these considerations do not apply to the immunocomplexes analysis which allows the discrimination of healthy subjects from cancer patients with enhanced specificity. The effects of the IC formation on the determination of the free tumor-associated antigens in serum or plasma are twofold: (i) When the antigen is involved in the CIC, it may be masked and thus not detected with a normal immunometric assay, this condition may explain the occurrence of false negative results. On the other hand, low levels of antigen released from normal cells may not form stable and persistent ICs. In the latter case, the free antigen may be detected leading to false positive results. Both sources of false results may be eliminated revealing the specific CIC as it is described in one embodiment of the following invention.

The diagnostic method devised by the applicant allows the simple determination of the tumor associated biomarker by means of specific reagents.

The research activity developed, on a large number of neoplastic diseases for which an antigenic biomarker is already known, led to the detection of immunocomplexes formed by said antigenic biomarker and autoantibody specific thereto. The detection of said specific complex is useful in the diagnosis for the given tumor. Said method for cancer diagnosis is based on the detection, in biological fluids, of specific CIC by contacting and incubating the samples obtained from the subject with specific reagents (SR) directed against the tumor associated antigen. Said method comprises the detection of the SR-CIC complex, where the presence of said complex indicates the presence of cancer. The SR considered comprise polyclonal and/or monoclonal antibodies or fragments thereof to be used to reveal, by immunometric methods, the presence of tumor markers involved in the formation of specific CIC and present in the blood of patients affected by neoplastic diseases. Non-limiting examples of said immunometric methods used to assess the CIC content in biological fluids comprise: direct, indirect or “sandwich” ELISAs (enzyme linked immunosorbent assays), radiometric assays (RIAs) or “sandwich” radiometric assays (IRMAs), or homogeneous immunometric assays exploiting different technologies such as “rapid-near-infrared spectroscopy”.

A typical example of immunoenzymatic methods for the detection in biological fluids of the specific CIC comprises:

1] Immobilization, on plastic microtiter plates for ELISA determinations or on particles to be used for diagnostic purposes, of specific reagents (SR) directed against the tumor marker in either the free or conjugated form;

2] Incubation of the biological fluid obtained from the patients with the immobilized compound;

3] Washing of the complex formed by the immobilized compound and the IC;

4] Incubation of said complex with polyclonal or monoclonal antibodies directed against antibodies of human origin, conjugated to traceable markers such as enzymes; chromogenic, fluorescent, radioactive, chemiluminescent compounds, coenzymes, or enzyme inhibitors;

5] Removal of the excess of antibody by suitable washing steps.

6] Detection of the adsorbed antibody by suitable reagents.

As used herein, biological fluids means: serum, plasma, lymphatic fluid, ascites, pleuric exudates, spinal fluid etc obtained from patients by conventional methods in amounts sufficient for performing assays thereon. These techniques are described in the literature and known by those skilled in the art.

Immunometric methods heretofore known are relative by nature. They do no allow the absolute determination of the concentration of an analyte of interest, but they allow the comparison of a signal proportional to said concentration with a similar signal obtained by using one or more samples of known concentration as calibrators. The immunometric methods of quantitative analysis cannot be devoided of calibration methodologies. In the clinical practice, and in diagnostic procedures, the analytes of interest are compounds the concentration thereof or their variation, are associated to the onset or the evolution of pathologic conditions. Among these compounds, there are species correlated to the development of neoplastic diseases, referred to as tumor markers or tumor associated antigens. Standard preparations of said compounds require their isolation from natural sources, and among them, biological material obtained from subjects affected by neoplastic diseases; alternatively, these species can be obtained exploiting recombinant DNA technologies. In the first case, the preparation of said calibrators requires handling and processing material of human origin, which poses serious supplying limitations and safety issues in the implementation of efficient industrial processes. In the second case the availability of recombinant products solve any supplying and safety problems, however, the expression of recombinant proteins is feasible only for molecular species of well defined composition and known sequence. There are however, analytes with relevance in the diagnosis of neoplastic diseases such as the aforementioned biomarker-autoantibody immunocomplexes, that are intrinsically heterogeneous and for them the expression as recombinant products is not practicable. At the present stage, the generation of calibrators for the quantitative analysis of these compounds, is possible only through extraction processes from samples of human origin. The introduction of synthetic or semi-synthetic processes for the obtainment of molecular species to be used as substitutes of said calibrators obtained by extraction from samples of human origin would simplify the productive processes increasing their safety standards reducing at the same time their costs and ensuring a better lot to lot reproducibility.

A first object of the present invention is a method for the synthesis of molecular species, hereinafter referred to as conjugates, displaying the reactivity of the biomarker-IgM immunocomplexes. The method of the present invention overcome all the problems related to the obtainment of biomarker-IgM immunocomplexes to be used as calibrators in diagnostic kits for cancer detection.

In Table 1, we report as a non-limiting example, biomarker and neoplasias of interest of the new method.

TABLE 1

| Tumor Marker | Cancer | Reference |
|---|---|---|
| Ki-67 | Astrocytoma | Br J Cancer 89(1): 128-34, 2003 |
| Fibronectin | Bladder cancer | Clin Chem Lab Med |
| | | 41(8): 1069-74, 2003 |
| Hepatoma up regulated protein | Bladder cancer | Anticancer Res 23(3B): 2729-33, |
| (HURP) | | 2003 |
| Mucin 7 (MUC7) | Bladder cancer | Urology 62(1): 182-86, 2003 |
| NMP22 | Bladder cancer | J Chin Med Assoc 66(5): 294- |
| | | 98, 2003 |
| NMP22 | Bladder cancer | Anticancer Res 23(2A): 805-12, |
| | | 2003 |
| Prostate stem cell antigen | Bladder cancer | J Urol 169(6): 2094-100, 2003 |
| (PSCA) | | |
| Telomerase | Bladder cancer | Urology 62(2): 362-67, 2003 |
| Tissue polypeptide antigen | Bladder cancer | Urology 62(2): 243-48, 2003 |
| (TPA) | | |
| CA 15-3, CA 27.29, AFP, CEA | Breast cancer | Biomed Sci Instrum 39: 408-14, |
| | | 2003 |
| CA 15-3, Ceruloplasmin, TPA | Breast cancer | East Afr Med J 77(6): 291-94, |
| | | 2000 |
| CEA, Cytokeratin 19 (CK19), | Breast cancer | Anticancer Res 23(2C): 1883-90, |
| Maspin | | 2003 |
| c-Met | Breast cancer | Breast Cancer Res 5(3): 71-76, |
| | | 2003 |
| Cytochrome P450 3A4 | Breast cancer | Biomed Sci Instrum 39: 24-29, |
| | | 2003 |
| Epithelial glycoprotein 2 | Breast cancer | Int J Cancer 106(4): 611-18, |
| (EGP2), Cytokeratin 19 (CK19) | | 2003 |
| MUC 1 | Breast cancer | Int J Biol Marker 15(4): 343-56, 2000 |
| Her2/Neu | Breast cancer | Clin Chem 49(10): 1579-98, |
| | | 2003 |
| MMP-9 | Breast cancer | Int J Cancer 106(5): 745-51, |
| | | 2003 |
| Human kallikrein 5 (hK5) | Breast cancer and ovarian canc [symbol] | Cancer Res 63(14): 3958-65, |
| | | 2003 |
| NMP179 | Cervical squamous epithelium c [symbol] | Anticancer Res 23(2A): 805-12, 2003 |
| CEA, Cytokeratin 19 (CK19), | Colorectal cancer | J Cancer Res Clin Oncol |
| Cytokeratin 20 (CK20) | | 129(3): 192-98, 2003 |
| Cytokeratin | Colorectal cancer | Colorectal Dis 5(2): 164-68, |
| | | 2003 |
| Cytokeratin 20 (CK20), CEA, | Colorectal cancer | Eur J Cancer 39(9): 1234-41, |
| Guanylyl cyclase C (GCC) | | 2003 |
| Her2/Neu | Colorectal cancer | Int J Cancer 105(6): 796-802, |
| | | 2003 |
| MUC6, MUC5AC | Colorectal cancer | Glycocong J 18(11-12): 907-14, |
| | | 2001 |
| RelA, NF-kb | Colorectal cancer | Oncology 65(1): 37-45, 2003 |
| Bcl-6, CD10 | B-cells lymphoma | Human Pathol 34(6): 610-16, |
| | | 2003 |
| CEA | Endometrial carcinoma | Anticancer Res 23(2A): 1103- |
| | | 06, 2003 |
| Cystein-rich fibroblast growth | Stomach cancer | Cancer Res 63(9): 2052-61, |
| factor receptor 1 (CFR-1) | | 2003 |
| p27, MIB-1 | Stomach cancer | Pathologica 95(1): 22-30, 2003 |
| Chromogranin A (CgA), | Gastrointestinal carcinoma | Cesk Pathol 39(2): 47-53, 2003 |
| Neuron specific enolase (NSE), | | |
| Synaptophysin, Leu-7, beta III- | | |
| tubulin | | |
| Cytokeratin, Epithelial | Head and neck carcinoma | Laryngoscope 113(5): 892-96, |
| membrane antigen (EMA) | | 2003 |
| Hyaluronidase (HYAL1) | Head and neck carcinoma | Int J Cancer 106(3): 438-45, |
| | | 2003 |
| Latent membrane protein 1 | Head and neck carcinoma | Cancer 97(8): 1909-13, 2003 |
| (LMP-1) | | |
| Alpha-fetoprotein (AFP), Des- | Hepatocellular carcinoma | Eur J Gastroenterol Hepatol |
| gamma-carboxy prothrombin | | 15(6): 641-48 |
| (DCP) | | |
| Cellular retinol binding protein | Hepatocellular carcinoma | Hepatology 38(2): 470-80, 2003 |
| 1 (CRBP1) | | |
| Glypcan-3 | Hepatocellular carcinoma | Gastroenterology 125(1): 89-97, |
| | | 2003 |
| Telomerase | Hepatocellular carcinoma | Oncology 64(4): 430-34, 2003 |
| Human cervical cancer | Hepatocellular carcinoma | Cancer Res 64, 5434-41, 2004 |
| oncogene (HCCR) | | |
| Osteopontin (OPN) | Hepatocellular carcinoma | Am J Gastroenterol 101: 2051- |
| | | 59, 2006 |
| Vascular endothelial growth | Hepatocellular carcinoma | J Gastroenterol 33(3): 376-82, |
| factor (VEGF) | | 1998 |
| Tissue inhibitor of | Hepatocellular carcinoma | Liver Int 24(4): 379-83, 2004 |
| metalloproteinases 1 (TIMP 1) | | |
| Survivin | Hepatocellular carcinoma | World J Gastroenterol |
| | | 13(46): 6264-8, 2007 |
| Heat shock protein 27 (HSP 27) | Hepatocellular carcinoma | Cancer 88(11): 2464-70, 2000 |
| Clusterin | Hepatocellular carcinoma | Human Pathol 35(11): 1340-6, 2004 |
| Preferentially expressed | Leukemia, Multiple myeloma | Leuk Lymphoma 44(3): 439-44, 2003 |
| antigen of melanoma (PRAME) | | |
| CEA, Chromogranin A, NSE, | Lung cancer | Anticancer Res 23(1A): 49-62, 2003 |
| Vascular endothelial growth | | |
| factor (VEGF), Stem cell factor | | |
| (SCF), Hepatocyte growth | | |
| factor/Scatter factor (HGF/SF) | | |
| Epidermal growth factor | Lung cancer | Int J Clin Oncol 8(2): 79-82, |
| receptor (EGFR) | | 2003 |
| M2-PK, CYFRA 21-1, NSE, | Lung cancer | Anticancer Res 23(2A): 899- |
| SCC | | 906, 2003 |
| Neuron specific enolase (NSE), | Melanoma | Magy Onkol 47(1): 89-104, |
| S-100B, Tyrosinase, LDH | | 2003 |
| Cytokeratin 20 (CK20), | Gastrointestinal cancer | Anticancer Res 23(3B): 2711-16, |
| Prostate stem cell antigen | | 2003 |
| (PSCA) | | |
| Medkine (MK) | Gastric carcinoma | BBRC 306(2): 329-32, 200 |
| CA 125 | Ovarian cancer | Oncology 65(1): 1-6, 2003 |
| CASA, CA 125 | Ovarian cancer | Anticancer Res 23(2A): 1115- |
| | | 18, 2003 |
| HE4 | Ovarian cancer | Adv Exp Med Biol 622: 15-21, |
| | | 2008 |
| Mesothelin | Ovarian cancer | Gynecol Oncol 99: 267-77, |
| | | 2005 |
| CA 19-9 | Pancreatic carcinoma | Anticancer Res 23(2A): 835-40, |
| | | 2003 |
| Survivin, p53, Bcl-2 | Pancreatic carcinoma | Int J Gastrointest Cancer 32(2- |
| | | 3): 73-81, 2002 |
| Secretogranin II-derived | Pheochromocytoma | J Clin Endocrinol Metab |
| peptide EM66 | | 88: 2579-85, 2003 |
| GLUT1, GLUT12 | Prostate cancer | Cancer 97(8): 2035-42, 2003 |
| Insulin-like growth factor | Prostate cancer | Virchows Arch 442(4): 329-35, |
| binding protein 2 (IGBFB2) | | 2003 |
| Prostate-specific antigen (PSA) | Prostate cancer | Ann Clin Biochem 38: 633-51, |
| | | 2001 |
| PSMA prostate-specific | Prostate cancer | Rev Urol 6 Suppl 10: S13-8 |
| membrane antigen (PSMA) | | |
| alpha-methylacyl-CoA | Prostate cancer | JAMA 287: 1662-1670, 2002 |
| racemase (AMACR) | | |
| Myogenin, MyoD1 | Rhabdomyosarcoma | J Clin Pathol 56(6): 412-16, |
| | | 2003 |
| Dipeptidyl Peptidase IV (DPP | thyroid cancer | Neoplasma 50(3): 159-64, 2003 |
| IV/CD 26) | (papillary carcinoma) | |
| Peroxisome proliferating | thyroid cancer | Am J Clin Pathol 120(2): 175- |
| activated receptor gamma | (papillary carcinoma) | 81, 2003 |
| (PPAR gamma) | | |

[symbol]

indicates data missing or illegible when filed

An embodiment of the present invention is a method useful in the determination of concentration ratios or mass ratios, in either an explicit or implicit form, as function of parameters, between at least two different biomolecules, functionalized or unfunctionalized fragments, functionalized or unfunctionalized sequences thereof (hereinafter referred to as non accessory components) involved in the presence or absence of at least a third molecule or biomolecule acting as a crosslinker or scaffold (hereinafter referred to as accessory component) in a single polydispersed or monodispersed molecular construct, hereinafter referred to as conjugate, with general formula AₐB_{b}C_{c} . . . Z_{z} endowed with the property to reproducibly maintain and display part or the whole of the chemical and immunochemical reactivity of their components considered separately. Said conjugate is endowed with the property of reproducibly displaying the reactivity of each ordered n-uple or pair, without lack of generality, of non accessory components when said reactivity is jointly assayed by immunometric methods. The method for the preparation of said bioconjugate comprises:

1] Functionalization, if required, of the species referred to as non accessory components

2] Functionalization, if required, of the species referred to as accessory components

3] Application of a combinatorial methodology for the identification of the best reaction conditions

4] Synthesis of the conjugate in the conditions identified in step 3
Step 1 requires the use of suitable reagents for the introduction of groups that are not present or are not sufficiently represented in the structure of the starting biomolecule. As used herein, “non accessory components” means species in which desired functional groups are introduced exploiting methods reported in the literature. As used herein, “functional groups” means any molecular moieties that are not present or are not sufficiently represented in the structure of the starting biomolecule. A non-limiting example of these groups is the following: thiols, pyridyl disulfides, azides, alkynes, alkenes, dienes, biotin, hydrazides hydrazine, aldehydes ketones, phenacyl halides and pseudohalides, primary alkyl halides and pseudohalides, secondary alkyl halides and pseudohalides, tertiary alkyl halides and pseudohalides, aromatic, benzylic, allylic halides and pseudohalides, maleimides, (His)x sequences. Conditions and methods are described in the literature and are known to those skilled in the art. As used herein, “functionalized non accessory components” also means biomolecules, fragments, sequences thereof comprising at least one of the following groups: thiol, amine, carboxy, hydroxyl, imidazole, phenoxy guanidinium, phenyl groups.
Step 2 requires the use of suitable reagents for the introduction of groups that are not present or are not sufficiently represented in the structure of the starting biomolecule. As used herein “functionalized accessory components” means amino acids polymers or other polymeric species in which functional groups are introduced exploiting methods reported in the literature. As used herein, “functional groups” means any molecular moieties that are not present or are not sufficiently represented in the structure of the starting biomolecule. A non-limiting example of these groups is the following: thiols, pyridyl disulfides, azides, alkynes, alkenes, dienes, biotin, hydrazides hydrazine, aldehydes ketones, phenacyl halides and pseudohalides, primary alkyl halides and pseudohalides, secondary alkyl halides and pseudohalides, tertiary alkyl halides and pseudohalides, aromatic, benzylic, allylic halides and pseudohalides, maleimides, (His)x sequences. Moreover, as used herein “accessory components” comprise homobifunctional cross-linkers. A non limiting example of homobifunctional cross-linkers is the following: active esters of dicarboxylic acids such as: suberic acid bis N-hydroxysuccinimide ester, sebacic acid bis N-hydroxysuccinimide ester, 3,3′-dithiodipropionic acid glutaraldehyde, 1,4-Bis[3-(2-pyridyldithio)propionamido]butane, 4,4′-diisothiocyanatestilbene-2,2′disulfonic acid disodium salt hydrate, adipic acid dihydrazide, bis[2-(4-azidosalicylamido)ethyl]disulfide, bis[2-(N-succinimidyl-oxycarbonyloxy)ethyl]sulfone, dimethyl 3,3′ -dithioproprionimidate dihydrochloride, dimethyl pimelimidate dihydrochloride, ethylene glycol-bis(succinic acid N-hydroxysuccinimide ester), bis(polyethylene glycol bis[imidazolyl carbonyl]).

As used herein “accessory components” also means heterobifunctional crosslinkers. A non limiting example of homobifunctional cross-linkers is the following: active esters of functionalized carboxylic acids such as N-hydroxysuccinimide esters of 3-(2-pyridyldithio)propionic acid, 3-maleimidobenzoic acid, 4-(4-maleimidophenyl)butyric acid, 4-(N-maleimidomethyl)cyclohexane-1-carboxylic acid, 4-maleimidobutyric acid, 5-azido-2-nitrobenzoic acid, 6-(4-azido-2-nitrophenylamino)hexanoic acid, 6-maleimidohexanoic acid, bromoacetic acid, iodoacetic acid, maleimidoacetic acid, S-acetylthioglycolic acid, maleimidoethyl succinic acid. Other bifunctional compounds such as 4-(N-maleimido)benzophenone, 4-azidophenacyl bromide, N-(3-dimethylaminopropyl)-N′-ethylcarbodiimide, N-((2-pyridyldithio)ethyl)-4-azidosalicylamide. As used herein “accessory components” means also proteins such as avidin, streptavidin, neutravidin, lectins, and other proteins endowed with the properties to selectively recognize those groups naturally present or artificially introduced in the structure of the non accessory components.,
Step 3 comprises.

1) a series of mixing operations using solutions of non accessory components as well as accessory components and buffer solutions. Said mixing operations are carried out in one or more reaction vessels, in the latter case, said reaction vessels are conveniently but not necessarily arranged in ordered arrays. The mixing operations are followed, if required, by one or more purification steps.

2) one or more immunometric procedures aimed to assess the reactivity of each ordered pair of non accessory components used in the conjugate synthesis.

For what concerns step 1), we consider, as a non limiting example, Q reaction vessels of given volume, conveniently arranged in an ordered array of m row and n columns (Q=m×n), conveniently but not necessarily constituted of a single object of inert material. In a typical example, each reaction compartment is loaded with a known concentration, A₀ of the protein species A. Said species may either be an accessory or non accessory component. The procedure requires a series of t (t=1, 2, 3, . . . , k) cycles comprising the delivery and mixing of suitable buffers or protein solutions in suitable buffers. Cycle t=1 require the following operations non necessarily carried out in this sequence.

a) according to the vertical direction of the reaction array, protein B is serially diluted. Said serial dilution is carried out according to a ratio defined as 2^{j1} (j1=0, 1, 2, . . . k where k<n−1) starting from the concentration B₀.

b) According to the horizontal direction of the reaction vessels array, a dilution of the species C is performed. Said species C may be either an accessory or a non accessory component. Said dilutions are performed according to the ratio 2ⁱ¹+hᵢ₁ (i1=0, 1, 2, . . . k where k<m−1 and hᵢ₁ is conveniently chosen so that |hᵢₗ|<2ⁱ¹) The initial concentration of species C is meant to be C₀.

To cycle t=2 correspond the following operations not necessarily carried out in this order:

a) according to the vertical direction of the reaction array, protein D is serially diluted. Said serial dilution is carried out according to a ratio defined as 2^{j2} (j1=0, 1, 2, . . . k where k<n−1) starting from the concentration D₀.

b) according to the horizontal direction of the reaction vessels array, a dilution of the species E is performed. Said species E may be either an accessory or a non accessory component. Said dilutions are performed according to the ratio 2ⁱ²+hᵢ₂ (i2=0, 1, 2, . . . , k where k<m−1 and hᵢ₂ is conveniently chosen so that |hᵢ₂|<2ⁱ²) The initial concentration of species E is meant to be E₀.

The process is continued until all the t cycles necessary to the delivery of all the protein solutions and buffers are completed. At the end of the process, in each reaction compartment qᵢⱼ the concentrations of all the species considered is the following: [δA₀, δB₀*2^{-j1}, δC₀*(2ⁱ¹+hᵢ₁)⁻¹, δD₀*2^{-j2}, δE₀*(2ⁱ²+hᵢ₂)⁻¹, . . . ], being δ the dilution factor.

At the end of the process, the content of each reaction compartment is assayed for its reactivity using immunometric methods.

For what concerns point 2) the selection of the best conditions require:

a) Immobilization, on plastic microtiter plates for ELISA determination, of monoclonal or polyclonal antibodies or Fab or F(ab′)₂ fragments thereof directed against one of the non accessory components comprised in the conjugate.

b) Incubation of the samples containing the conjugate of interest with the immobilized antibodies or fragments thereof.

c) Washing of the complex formed between the conjugate and the immobilized antibody or fragment thereof.

d) Detection of the presence of the antibody-conjugate complex with the aid of reagents selective or specific for one of the non accessory components different from that involved in the interaction between the conjugate and the immobilized antibody or fragment thereof.

Identification of the reaction compartments containing conjugates with the desired reactivity and identification of the reaction parameters t, jt, it, hᵢₜ δ, A₀, B₀, . . . Z₀ corresponding to those qᵢⱼ compartment containing the conjugates displaying the desired reactivity.

All these methods are known by those skilled in the art.
Step 4 require the synthesis of the conjugate identified during the phase 3.

The preferred method for the preparation of the conjugate comprises:

1] functionalization by biotinylation of the species collectively described as non accessory components

2] use of avidin, streptavidin or neutravidin as accessory component.

3]application of a combinatorial methodology for the determination of the best reaction conditions for the obtainment of conjugates with the desired reactivity.

4] synthesis of the conjugates according to the conditions identified in said combinatorial screening.

The present invention will be described by some non-limiting examples:

### EXAMPLE 1

### Conjugate Displaying the reactivity of IgM and SCCA

SCCA and IgM were biotinylated using biotinamidohexanoyl-6-aminohexanoic acid N-hydroxysuccinimide ester (Sigma-Aldrich B3295-50MG) and extensively dialyzed against PBS. The chemistry and the conditions used in the biotinylation process are known to those skilled in the art. A microtiter plate for ELISA determination (Greiner, Medium binding STRIP PLATE) was treated with a 5% solution of dried skimmed milk in PBS (PBS-M) (300 μl/well). After 12 hours of incubation at 4° C. the plate was washed with PBS containing 0.05% Tween 20 (PBS-T). Each well was loaded with the following accessory and non accessory components:

a) Avidin (Applichem) 0-20 μg in PBS

b) Biotinylated IgM, 2.5-40 μg, preferably 10 μg, biotinylated SCCA with concentration comprised in the range 20-0.05 μg/ml.

The plate was allowed to stand at room temperature for 1 hour. After the incubation time, the content of each well was tested to assess the reactivity due to the conjugate. An ELISA microtiter plate (Greiner 762071, High binding STRIP PLATE) was treated with 100 μl/well of polyclonal rabbit anti-SCCA antibody (Xeptagen) at the final concentration of 10 μg/ml and incubated at 4° C. in a humid chamber for 12 hours. The plate was washed 3 times with 300 μl/well of PBS containing 0.05% Tween 20 (PBS-T) (Sigma-Aldrich P-1379), each well was filled with 300 μl of PBS containing 3% bovine serum albumin (Sigma-Aldrich A4503-1KG) and incubated at room temperature for 2 hours in a humid closed chamber. The plate was washed 3 times with PBS-T (300 μl/well), in each well 100 μl of the conjugate solutions were loaded and the plate was incubated for 1 hour at room temperature. The plate was washed with PBS-T buffer 6×300 μl/well and each well was loaded with 100 μl of anti-human IgM-HRP conjugated antibody (Sigma-Aldrich A0420-1ML) diluted 1000 times, the plate was incubated for 1 hour. The plate was washed with PBS-T buffer 6×300 μl/well and each well was loaded with 150 μl of ABTS [2,2′-azino-bis(3-ethylbenzthiazoline-6-sulphonic acid)] 0.2 mg/ml in phosphate-citrate buffer pH 5.0 containing 5 mM hydrogen peroxide. The plate was incubated at 37±2° C. for 20 minutes, the optical density was measured at 405 nm with a suitable microtiter plate reader. The conditions corresponding to the most reactive preparations were used for the preparative scale synthesis of the conjugate.

### EXAMPLE 2

### Conjugate Displaying the Reactivity of the CEA-IgM Immunocomplex

CEA and IgM were biotinylated using biotinamidohexanoyl-6-aminohexanoic acid N-hydroxysuccinimide ester (Sigma-Aldrich B3295-50MG) and extensively dialyzed against PBS. The chemistry and the conditions used in the biotinylation process are known to those skilled in the art. A microtiter plate for ELISA determination (Greiner, Medium binding STRIP PLATE) was treated with a 5% solution of dried skimmed milk in PBS (PBS-M) (300 μl/well). After 12 hours the plate was washed with PBS containing 0.05% Tween 20 (PBS-T). Each well was loaded with the following accessory and non accessory components:

a) Biotinylated IgM, 2.5-40 μg, preferably 10 μg, dissolved in PBS buffer

b) Biotinylated human CEA with concentration comprised in the range 20-0.05 μg/ml in PBS, Avidin (Applichem) in the range 0-20 μg/well in PBS.

The plate was allowed to stand at room temperature for 1 hour. After the incubation time, the content of each well was tested to assess the reactivity due to the conjugate. An ELISA microtiter plate (Greiner 762071, High binding STRIP PLATE) was treated with 100 μl/well of polyclonal rabbit anti-CEA antibody (Dako A0115) at the final concentration of 10 μ/ml and incubated at 4° C. in a humid chamber for 12 hours. The plate was washed 3 times with 300 μl/well of PBS containing 0.05% Tween 20 (PBS-T) (Sigma-Aldrich P-1379), each well was filled with 300 μl of PBS containing 3% bovine serum albumin (Sigma-Aldrich A4503-1KG) and incubated at room temperature for 2 hours in a humid closed chamber. The plate was washed 3 times with PBS-T (300 μl/well), in each well 100 μl of the conjugate solutions were loaded and the plate was incubated for 1 hour at room temperature. The plate was washed with PBS-T buffer 6×300 μl/well and each well was loaded with 100 μl of a 10 μl/ml solution of anti-human IgM-HRP conjugated antibody (Sigma-Aldrich A0420-1ML) diluted 1000 times, the plate was incubated for 1 hour. The plate was washed with PBS-T buffer 6×300 μl/well and each well was loaded with 150 μl ABTS [2,2′-azino-bis(3-ethylbenzthiazoline-6-sulphonic acid)] 0.2 mg/ml in phosphate-citrate buffer pH 5.0 containing 5 mM hydrogen peroxide.

The plate was incubated at 37±2° C. for 20 minutes, the optical density was measured at 405 nm with a suitable microtiter plate reader. The conditions corresponding to the most reactive preparations were used for the preparative scale synthesis of the conjugate.

### EXAMPLE 3

### Conjugate Displaying the Reactivity of the Ordered Pairs of Proteins (SCCA-IgM) (CEA-IgM), (SCCA-CEA)

CEA, SCCA and IgM were biotinylated using biotinamidohexanoyl-6-aminohexanoic acid N-hydroxysuccinimide ester (Sigma-Aldrich B3295-50MG) and extensively dialyzed against PBS. The chemistry and the conditions used in the biotinylation process are known to those skilled in the art. A microtiter plate for ELISA determination (Greiner, Medium binding STRIP PLATE) was treated with a 5% solution of dried skimmed milk in PBS (PBS-M) (300 μl/well). After 12 hours the plate was washed with PBS containing 0.05% Tween 20 (PBS-T). Each well was loaded with the following accessory and non accessory components:

a) Avidin (Applichem) 0-20 μg in PBS

b) Biotinylated IgM, 2.5-40 μg, preferably 10 μg, biotinylated SCCA concentration comprised in the range 20-0.05 μg/ml, biotinylated CEA in concentration comprised in the range 20-0.05 μg/ml in PBS. The plate was allowed to stand at room temperature for 1 hour. After the incubation time, the content of each well was tested to assess the reactivity due to the conjugate.

### Screening for the Reactivity of the Ordered Pair CEA-IgM

A microtiter ELISA plate (Greiner 762071, High binding STRIP PLATE) was treated with 100 μl/well of polyclonal rabbit anti-CEA antibody (Dako A0115) at the final concentration of 10 μg/ml and incubated at 4° C. in a humid chamber for 12 hours. The plate was washed 3 times with 300 μl/well of PBS containing 0.05% Tween 20 (PBS-T) (Sigma-Aldrich P-1379), each well was filled with 300 μl of PBS containing 3% bovine serum albumin (Sigma-Aldrich A4503-1KG) and incubated at room temperature for 2 hours in a humid closed chamber.

The plate was washed 3 times with PBS-T (300 μl/well), in each well 100 μl of the conjugate solutions were loaded and the plate was incubated for 1 hour at room temperature. The plate was washed with PBS-T buffer 6×300 μl/well and each well was loaded with 100 μl of anti-human IgM-HRP conjugated antibody solution (Sigma-Aldrich A0420-1ML) diluted 1000 times, the plate was incubated for 1 hour. The plate was washed with PBS-T buffer 6×300 μl/well and each well was loaded with 150 μl of ABTS [2,2′-azino-bis(3-ethylbenzthiazoline-6-sulphonic acid)] 0.2 mg/ml in phosphate-citrate buffer pH 5.0 containing 5 mM hydrogen peroxide. The plate was incubated at 37±2° C. for 20 minutes, the optical density was measured at 405 nm with a suitable microtiter plate reader.

### Screening for the Reactivity of the Ordered Pair SCCA-IgM

A second ELISA microtiter plate (Greiner 762071, High binding STRIP PLATE) was treated with 100 μl/well of polyclonal rabbit anti-SCCA antibody (Xeptagen) at the final concentration of 10 μg/ml and incubated at 4° C. in a humid chamber for 12 hours. The plate was washed 3 times with 300 μl/well of PBS containing 0.05% Tween 20 (PBS-T) (Sigma-Aldrich P-1379), each well was filled with 300 μl of PBS containing 3% bovine serum albumin (Sigma-Aldrich A4503-1KG) and incubated at room temperature for 2 hours in a humid closed chamber. The plate was washed 3 times with PBS-T (300 μl/well), in each well 100 μl of the conjugate solutions were loaded and the plate was incubated for 1 hour at room temperature. The plate was washed with PBS-T buffer 6×300 μl/well and each well was loaded with 100 μl of solution of anti-human IgM-HRP conjugated antibody (Sigma-Aldrich A0420-1ML) diluted 1000 times, the plate was incubated for 1 hour. The plate was washed with PBS-T buffer 6×300 μl/well and each well was loaded with 150 μl of ABTS [2,2′-azino-bis(3-ethylbenzthiazoline-6-sulphonic acid)] 0.2 mg/ml in phosphate-citrate buffer pH 5.0 containing 5 mM hydrogen peroxide. The plate was incubated at 37±2° C. for 20 minutes, the optical density was measured at 405 nm with a suitable microtiter plate reader.

### Screening for the Reactivity of the Ordered Pair SCCA-CEA

A third microtiter plate for ELISA determinations (Greiner 762071, High binding STRIP PLATE) was treated with 100 μl/well of monoclonal mouse anti-SCCA antibody (Xeptagen) at the final concentration of 10 μg/ml and incubated at 4° C. in a humid chamber for 12 hours. The plate was washed 3 times with 300 μl/well of PBS containing 0.05% Tween 20 (PBS-T) (Sigma-Aldrich P-1379), each well was filled with 300 μl of PBS containing 3% bovine serum albumin (Sigma-Aldrich A4503-1KG) and incubated at room temperature for 2 hours in a humid closed chamber. The plate was washed 3 times with PBS-T (300 μl/well), in each well 100 μl of conjugate solutions were loaded and the plates incubated for 1 hour at room temperature. The plate was washed with PBS-T buffer 6×300 μl/well and each well was loaded with 100 μl of a 2 μg/ml solution of polyclonal rabbit anti-CEA antibody (Dako A0115) in PBS-T containing 1% BSA, the plate was incubated for 1 hour. The plate was washed with PBS-T buffer 6×300 μl/well and each well was loaded with 100 μl of a solution of goat anti-rabbit IgG-HRP conjugated antibody (KPL 474-1506), the plate was incubated for 1 hour. The plate was washed with PBS-T buffer 6×300 μl/well and each well was filled with 150 μl of ABTS [2,2′-azino-bis(3-ethylbenzthiazoline-6-sulphonic acid)] 0.2 mg/ml in phosphate-citrate buffer pH 5.0 containing 5 mM hydrogen peroxide. The plate was incubated at 37±2° C. for 20 minutes, the optical density was measured at 405 nm with a suitable microtiter plate reader.

### EXAMPLE 4

### Conjugate Displaying the Reactivity of the AFP-IgM Immunocomplex

AFP and IgM were biotinylated using biotinamidohexanoyl-6-aminohexanoic acid N-hydroxysuccinimide ester (Sigma-Aldrich B3295-50MG) and extensively dialyzed against PBS. The chemistry and the conditions used in the biotinylation process are known to those skilled in the art. A microtiter plate for ELISA determination (Greiner, Medium binding STRIP PLATE) was treated with a 5% solution of dried skimmed milk in PBS (PBS-M) (300 μl/well). After 12 hours the plate was washed with PBS containing 0.05% Tween 20 (PBS-T). Each well was loaded with the following accessory and non accessory components:

a) Avidin (Applichem) 0-20 μg in PBS

b) Biotinylated IgM, 2.5-40 μg, preferably 10 μg, biotinylated AFP with concentration comprised in the range 20-0.05 μg/ml.

The plate was allowed to stand at room temperature for 1 hour. After the incubation time, the content of each well was tested to assess the reactivity due to the conjugate. An ELISA microtiter plate (Greiner 762071, High binding STRIP PLATE) was treated with 100 μl/well of polyclonal rabbit anti-AFP antibody (Dako A008) at the final concentration of 10 μg/ml and incubated at 4° C. in a humid chamber for 12 hours. The plate was washed 3 times with 300 μl/well of PBS containing 0.05% Tween 20 (PBS-T) (Sigma-Aldrich P-1379), each well was filled with 300 μl of PBS containing 3% bovine serum albumin (Sigma-Aldrich A4503-1KG) and incubated at room temperature for 2 hours in a humid closed chamber. The plate was washed 3 times with PBS-T (300 μl/well), in each well 100 μl of the conjugate solutions were loaded and the plate incubated for 1 hour at room temperature. The plate was washed with PBS-T buffer 6×300 μl/well and each well was loaded with 100 μl of anti-human IgM-HRP conjugated antibody solution (Sigma-Aldrich A0420-1ML) diluted 1000 times, the plate was incubated for 1 hour. The plate was washed with PBS-T buffer 6×300 μl/well and each well was loaded with 150 μl of ABTS [2,2′-azino-bis(3-ethylbenzthiazoline-6-sulphonic acid)] 0.2 mg/ml in phosphate-citrate buffer pH 5.0 containing 5 mM hydrogen peroxide. The plate was incubated at 37±2° C. for 20 minutes, the optical density was measured at 405 nm with a suitable microtiter plate reader. The conditions corresponding to the most reactive preparations were used for the preparative scale synthesis of the conjugate.

### EXAMPLE 5

### Quantitative Determination of the Specific ICC Containing the Protein SCCA1 and/or its Variants and the Auto-Antibody Specific to them in Serum from Hepatocellular Carcinoma (HCC) Using as Calibrator the Object of the Following Invention

A microplate for ELISA determinations (Greiner 762071, High binding STRIP PLATE) was treated with 100 μl of a solution of rabbit polyclonal anti-SCCA antibody (Xeptagen) in PBS buffer for 12 hours at 4° C. The plate was washed with PBS containing 0.05% Tween 20. To each well were added 300 μl of a 1% solution of bovine serum albumin in PBS (BSA, Sigma-Aldrich, A-4503) (PBS-1% BSA). After a 2 hours incubation at room temperature, the plate was washed 6 times with PBS-T, and filled with 100 μl of human serum at different dilutions. In the same plate, a series of wells was used to construct the calibration curve for the specific CIC SCCA-IgM using the calibrator prepared according to the example 1. The concentration range spanned was between 1000-15 arbitrary units per milliliter (AU/ml). The plate was incubated for 1 hour at room temperature. The plate was washed 6 times with a solution of PBS-T and filled with 100 μl of a solution of polyclonal antibody, directed against human immunoglobulins, conjugated to horseradish peroxidase diluted 1000 times and incubated for 1 hour at room temperature. At the end of the incubation time, the plate was washed 6 times with a solution of PBS-T and filled with 200 μl of a solution of chromogenic substrate ABTS [2,2′-azino-bis(3-ethylbenzthiazoline-6-sulphonic acid)] 0.2 mg/ml in phosphate-citrate buffer pH 5.0 containing 5 mM hydrogen peroxide. After incubation at various times (20-30 minutes) at 37° C., the absorbance at 405 nm was read using a suitable plate reader (Biorad). The concentration of SCCA-IgM in the samples were determined by interpolation of the absorbance values on the standard curve. The analysis of sera from HCC patients according to the previously described method confirmed the presence of the specific CIC, while the control sera obtained from healthy subjects resulted to be negative. (100% specificity).

Hence, with reference to the above discussion and the attached table,

What we claim is:

1. A method for the synthesis of molecular species or conjugates endowed with the capability to allow the explicit or implicit determination of concentration ratios or mass ratios between at least two different molecules or biomolecules conveniently functionalized, fragments or sequences thereof, or non accessory components; being included in the presence or absence of a third molecule or biomolecule acting as a cross-linker, scaffold or accessory component, in a monodispersed or polydispersed molecular object or conjugate of general formula AₐB_{b}C_{c} . . . Z_{z}. Said method is endowed with the following properties: (i) the products obtained according to it display in reproducible manner part or the whole of the immunoreactivity of its non accessory or accessory components, (ii) said method allow to reproducibly optimize the reactivity of any ordered n-uple of non accessory components independently from the intrinsic reactivity of said components, when said reactivity is tested jointly.

2. Method of claim 1 characterized by the fact that the concentration or mass ratios are explicitly or implicitly defined as a set of parameters.

3. Method of claim 1, 2 characterized by the fact that at least one of the non accessory components considered belongs to the class of human immunoglobulins such as IgG, IgA, IgE, IgM, IgD, preferably IgM, fragments or sequences thereof.

4. Method of claims 1, 2 characterized by the fact that at least one of the non accessory components considered belongs to the class of biomolecules relevant as selective or specific markers for the onset or progression of pathologic conditions.

5. Method of claim 4 characterized by the fact that said biomolecule is a tumor marker.

6. Method of claim 4 or 5, characterized by the fact that said biomolecules do not comprise those proteins able to react under turnover conditions with reagents of protein or non protein nature which release themselves or by interaction with other species, chromophoric, fluorophoric, chemiluminescent substances. Instead, said biomolecules may comprise enzymes or complexes containing enzymes of relevance as tumor markers.

7. Method of claims 1, 3, 4, 5 characterized by the fact that at least one of the accessory components is endowed with the property to present a chemical reactivity, naturally present or artificially introduced, complementary to that due to the groups naturally present or artificially introduced in the molecules referred to as non accessory components.

8. Immunometric method relevant to diagnostic procedures characterized by the fact that it uses said conjugates obtained applying the method of one or more of the above reported claims.

9. Immunometric method of claim 8 characterized by the fact of being immunoenzymatic.

10. Immunometric methodologies relevant to diagnostic procedures as stated in claim 9, characterized by the fact that said immunoenzymatic methods are ELISA assays.

11. Immunometric method relevant to diagnostic procedures as of claim 10, characterized by the fact that said ELISA assays are used to determine the concentration of species pertinent to the diagnosis of neoplastic diseases or other properties correlated thereto.

12. Immunometric methodology relevant to diagnostic procedures as of claim 11, characterized by the fact that said species are immunocomplexes biomarker-autoantibody.

13. Immunometric methodology as of claim 12, characterized by the fact that said autoantibodies are IgM.

14. Immunometric methodology relevant to diagnostic procedures as of claim 13 characterized by the fact that it associates one or more of the following markers for the respective neoplastic diseases:
Ki-67 for Astrocytoma;
Fibronectin, Hepatoma up regulated protein (HURP), Mucin 7 (MUC7), NMP22, NMP22, Prostate stem cell antigen (PSCA), Telomerase, Tissue polypeptide antigen (TPA) for bladder cancer;
CA 15-3, CA 27.29, AFP, CEA, CA 15-3, Ceruloplasmin, TPA, CEA, Cytokeratin 19 (CK19), Maspin, c-Met Cytochrome P450 3A4, Epithelial glycoprotein 2 (EGP2), Cytokeratin 19 (Ck 19), ErbB-2 Her2/Neu MMP-9 for breast cancer;
Human kallikrein 5 (hK5) for breast and ovarian cancer;
NMP 179 for the cervical squamous epithelium cancer;
CEA, Cytokeratin 19 (CK19), Cytokeratin 20 (CK20), Cytokeratin, Cytokeratin 20 (CK20), CEA, Guanylyl cyclase C (GCC), Her2/Neu, MUC6, MUC5AC, RelA, NF-kb, for colorectal cancer;
Bcl-6, CD10, for B-cells lymphoma;
CEA, for endometrial carcinoma;
Cystein-rich fibroblast growth factor receptor 1 (CFR-1)m p27, MIB-1 for stomach cancer;
Chromogranin A (CgA), Neuron specific enolase (NSE), Synaptophysin, Leu-7, beta III-tubulin for gastrointestinal carcinoma;
Cytokeratin, Epithelial membrane antigen (EMA), Hyaluronidase (HYAL1), Latent membrane protein 1 (LMP-1) for head and neck carcinoma;
Alpha-fetoprotein (AFP), Des-gamma-carboxy prothrombin (DCP) for hepatocellular carcinoma;
Cellular retinol binding protein 1 (CRBP1), Glypcan-3, Telomerase for hepatocellular carcinoma;
Preferentially expressed antigen of melanoma (PRAME) for leukemia and multiple myeloma;
CEA, Chromogranin A, NSE, Vascular endothelial growth factor (VEGF), Stem cell factor (SCF), Hepatocyte growth factor/Scatter factor (HGF/SF) for lung cancer;
Epidermal growth factor receptor (EGFR), M2-PK, CYFRA 21-1, NSE, SCC, for lung cancer;
Neuron specific enolase (NSE), S-100B, Tyrosinase, LDH, for melanoma;
Cytokeratin 20 (CK20), Prostate stem cell antigen (PSCA) for gastrointestinal cancer;
Medicine (MK) for gastric carcinoma;
CA 125, CASA, CA 125 for ovarian cancer;
CA 19-9 for pancreatic carcinoma;
Survivin, p53, Bcl-2 for pancreatic carcinoma;
Secretogranin II-derived peptide EM66 for pheochromocytoma;
GLUT1, GLUT12, Insulin-like growth factor binding protein 2 (IGBFB2) for prostate cancer;
Myogenin, MyoD1 for rhabdomyosarcoma;
Dipeptidyl Peptidase IV (DPP IV/CD 26) for thyroid cancer (papillary carcinoma);
Peroxisome proliferating activated receptor gamma (PPAR gamma) for thyroid cancer (papillary carcinoma).
